(19) United States
(12) Patent Application Publication
(10) Pub. No.: US 2011/0207784 A1
(43) Pub. Date: Aug. 25, 2011

(54) OXADIAZOLE-2-OXIDES AS ANTISCHISTOSOMAL AGENTS

(75) Inventors: Craig J. Thomas, Gaithersburg, MD (US); David J. Maloney, Point of Rocks, MD (US); Ganesha Rai Bantukallu, Arlington, VA (US); Ahmed A. Sayed, Cairo (EG); Anton Simeonov, Bethesda, MD (US); David L. Williams, Oak Park, IL (US)

(73) Assignees: THE UNITED STATES OF AMERICA, AS REPRESENTED BY THE SECRETARY,; DEPARTMENT OF HEALTH AND HUMAN SERVICES, BETHESDA, MD (US); ILLINOIS STATE UNIVERSITY, Normal, IL (US)

(21) Appl. No.: 13/057,667

(22) PCT Filed: Aug. 13, 2009
(86) PCT No.: PCT/US09/53715

§ 371 Date: May 12, 2011

Related U.S. Application Data
(60) Provisional application No. 61/088,970, filed on Aug. 14, 2008.

(51) Int. Cl. A61K 31/4245; C07D 271/08; C07D 413/10; C07D 413/04; C07D 413/14; A61P 33/12

(52) U.S. Cl. 514/364; 548/125

(57) ABSTRACT

The invention provides 1,2,5-oxadiazole-containing compounds of Formula (I), wherein R¹, A, and R² are as defined herein, that are useful in treating schistosomiasis. The invention also provides a composition comprising a pharmaceutically suitable carrier and at least one compound of the invention, and a method of treating schistosomiasis in a mammal.

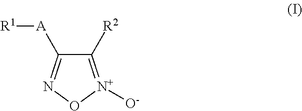

### CROSS-REFERENCE TO A RELATED APPLICATION

This application claims the benefit of U.S. Provisional Patent Application No. 61/088,970, filed Aug. 14, 2008, the disclosure of which is incorporated by reference.

### BACKGROUND OF THE INVENTION

Schistosomiasis is a chronic disease caused by the trematode flatworm of the genus Schistosoma, of which Schistosoma mansoni, Schistosoma japonicum, and Schistosoma haematobium are the most important. The disease remains a major, neglected health problem in many tropical areas. The health burden resulting from schisosomiasis is estimated to include more than 200 million people infected, 779 million at risk of infection, 280,000 deaths annually, and more than 20 million individuals experiencing high morbidity. Clinical manifestations of schistosomiasis infection include abdominal pain, cough, diarrhea, eosinophilia, fever, fatigue, and hepatosplenomegaly.

The primary route of infection occurs through contact with infected river and lake water, at which time the parasite burrows into the skin, matures, then migrates to other areas of the body. Adult S. mansoni parasites reside in the mesenteric veins of their human hosts, where they can survive for up to 30 years.

The need to control schistosomiasis is acute and efforts have been ongoing for years on three main fronts: prevention (via establishment and maintenance of sources of safe potable water), development of a vaccine, and use of drugs to treat the infection. Although the number of schistosomiasis cases worldwide is enormous, the number of drugs available to treat the disease is small. Earlier in the twentieth century, schistosomiasis was treated with highly toxic antimonial compounds, of which the most common was potassium antimonyl tartrate. During the past three decades, the only drug used against the infection is praziquantel, which is administered orally, is stable, effective against all major schistosome species in a single dose, and is relatively inexpensive (see, e.g., Cioli et al, Parasitol. Res. 90 Supp. I, 83-9 (2003); Doenhoff et al., Parasitol. Today 16, 364-366 (2000)). However, because of high reinfection rates, praziquantel must be administered on an annual or semi-annual basis. Preliminary reports of praziquantel-resistant cases, and the generation of praziquantel-resistant parasites in the laboratory, highlight the need for new drugs to treat the disease.

Arteminisinin has shown promise as a new drug for the treatment of schistosomiasis, although its use therefor may be restricted in areas of malaria transmission so that its use as an antimalarial is not put at risk (see., e.g., Utzinger et al., Curr. Op. Inv. Drugs 8, 104-116 (2007)). Simplified derivatives of artemisinin, the 1,2,4-trioxolanes, show promise and potential selectivity, but these, like the parent compound, are significantly less active against adult schistosome parasites. Oxamniquine, a tetrahydroquinoline derivative, is effective only against S. mansoni and resistance has been reported, further reducing its potential value in schistosomiasis control (see, e.g., Cioli et al., Pharmacol. Therapeutics 68, 35-85 (1995)).

In view of the foregoing, there is a desire to provide new compounds for use in the treatment against schistosomiasis.

### BRIEF SUMMARY OF THE INVENTION

The present invention provides compounds that are potent inhibitors of TGR (thioredoxin glutathione reductase—a critical parasite redox protein). In addition, the present invention provides compositions comprising these compounds and methods of using these compounds as therapeutic agents in the treatment of schistosomiasis.

In an embodiment, the invention provides a compound of the formula (I):

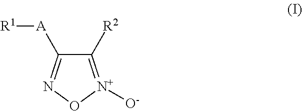

wherein A is selected from the group consisting of a bond, —C(═O)—, —C(═NR³)—, and —C(═NOR³)—,

R¹ is selected from the group consisting of a C₆-C₁₀ aryl group, a heterocycloaryl group, and R⁶, each optionally substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, C₆-C₁₀ heterocycloaryl, 3-cyano-1,2,5-oxadiazol-4-yl-2-oxide, C₁-C₆ haloalkyl, C₁-C₆ dihaloalkyl, C₁-C₆ trihaloalkyl, —NO₂, —OH, —OR⁴, —SH, —SR⁴, —SOR⁴, —SO₂R⁴, —COR⁴, —COOH, —COOR⁴, —CONHR⁴, and —CONHR⁴R⁵,

R² is selected from the group consisting of C₁-C₆ alkyl, C₂-C₆ alkenyl, C₂-C₆ alkynyl, C₃-C₈ cycloalkyl, C₃-C₈ cycloalkenyl, —CH₂OH, —CHO, —COOH, —CONH₂, —C═NR⁵, —C═NOH, —C═NOR⁵, and —CN,

R³, R⁴, and R⁵ are selected from the group consisting of C₁-C₆ alkyl, C₂-C₆ alkenyl, C₂-C₆ alkynyl, C₃-C₈ cycloalkyl, and C₃-C₈ cycloalkenyl, and

R⁶ is methylenedioxyphenyl, 2,3-benzofuranyl, or 2,3-dihydrobenzofuranyl,

or a pharmaceutically acceptable salt thereof.

The invention also provides a pharmaceutical composition comprising a compound or salt of the invention and a pharmaceutically acceptable carrier.

The invention further provides a method for treating schistomasiasis in a mammal comprising administering an effective amount of the compound on the invention to a mammal afflicted therewith.

### BRIEF DESCRIPTION OF THE SEVERAL VIEWS OF THE DRAWINGS

FIG. 1 illustrates a synthetic scheme to prepare oxadiazole-2-oxide compounds in accordance with an embodiment of the invention.

FIG. 2 illustrates a synthetic scheme to prepare oxadiazole-2-oxide compounds in accordance with another embodiment of the invention.

### DETAILED DESCRIPTION OF THE INVENTION

In accordance with an embodiment, the invention provides a compound of the formula (I):

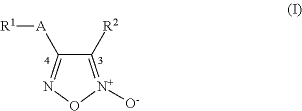

wherein A is selected from the group consisting of a bond, —C(═O)—, —C(═NR³)—, and —C(═NOR³)—,

R¹ is selected from the group consisting of a C₆-C₁₀ aryl group, a heterocycloaryl group, and R⁶, each optionally substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, C₆-C₁₀ heterocycloaryl, 3-cyano-1,2,5-oxadiazol-4-yl-2-oxide, C₁-C₆ haloalkyl, C₁-C₆ dihaloalkyl, C₁-C₆ trihaloalkyl, —NO₂, —OH, —OR⁴, —SH, —SR⁴, —SOR⁴, —SO₂R⁴, —COR⁴, —COOH, —COOR⁴, —CONHR⁴, and —CONHR⁴R⁵,

R² is selected from the group consisting of C₁-C₆ alkyl, C₂-C₆ alkenyl, C₂-C₆ alkynyl, C₃-C₈ cycloalkyl, C₃-C₈ cycloalkenyl, —CH₂OH, —CHO, —COOH, —CONH₂, —C═NR⁵, —C═NOH, —C═NOR⁵, and —CN,

R³, R⁴, and R⁵ are selected from the group consisting of C₁-C₆ alkyl, C₂-C₆ alkenyl, C₂-C₆ alkynyl, C₃-C₈ cycloalkyl, and C₃-C₈ cycloalkenyl, and

R⁶ is methylenedioxyphenyl, 2,3-benzofuranyl, or 2,3-dihydrobenzofuranyl,

with the proviso that when A is a bond and R² is CN or CONH₂, R¹ is not unsubstituted aryl,

or a pharmaceutically acceptable salt thereof.

In accordance with an embodiment, the group A represents a bond. The bond is a single bond between the substituent R¹ and the 4-position of the oxadiazole ring.

In certain embodiments, R² is selected from the group consisting of —CH₂OH, —CHO, —C═NR⁵, —C═NOH, —C═NOR⁵, and —CN, wherein R⁵ is selected from the group consisting of C₁-C₆ alkyl, C₂-C₆ alkenyl, C₂-C₆ alkynyl, C₃-C₈ cycloalkyl, and C₃-C₈ cycloalkenyl. In a preferred embodiment, R² is selected from the group consisting of —CHO, —C═NOH, —C═NOR⁵, and —CN. More preferably, R² is —CN.

In any of the embodiments above, R¹ is a C₆-C₁₀ aryl group, which may be unsubstituted or substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, 3-cyano-1,2,5-oxadiazol-4-yl-2-oxide, C₁-C₆ haloalkyl, C₁-C₆ dihaloalkyl, C₁-C₆ trihaloalkyl, —NO₂, —OH, —OR⁴, —SH, —SR⁴, —COR⁴, —COOR⁴, —CONHR⁴, and —CONHR⁴R⁵, wherein R³, R⁴, and R⁵ are selected from the group consisting of C₁-C₆ alkyl, C₂-C₆ alkenyl, C₂-C₆ alkynyl, C₃-C₈ cycloalkyl, and C₃-C₈ cycloalkenyl. The C₆-C₁₀ aryl group can be a phenyl group or a naphthyl group. When the C₆-C₁₀ aryl group is a naphthyl group, the naphthyl group can be attached to the oxadiazole at the 1-position or the 2-position of the naphthyl group. In a preferred embodiment, R¹ is a phenyl group substituted by 1, 2, 3, 4, or 5 substituents selected from the group consisting of halo, C₁-C₆ trihaloalkyl, —NO₂, —OH, and —OR⁵. The C₆-C₁₀ aryl group can be substituted at any available position on the aryl ring system.

In certain embodiments, R¹ is a C₆-C₁₀ aryl group substituted by 1, 2, 3, 4, or 5 halo, nitro, C₁-C₆ trihaloalkyl, hydroxyl, and —OR⁵ substituents. Non-limiting examples of halo substituted C₆-C₁₀ aryl groups include 2-fluorophenyl, 3-fluorophenyl, 4-fluorophenyl, 2,3-difluorophenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,6-trifluorophenyl, 3,4,5-trifluorophenyl, as well as the chloro, bromo, and iodo analogs thereof. Non-limiting examples of nitro substituted C₆-C₁₀ aryl groups include 2-nitrophenyl, 3-nitrophenyl, 4-nitrophenyl, 2,4-dinitrophenyl, 2,5-dinitrophenyl, 2,6-dinitrophenyl, and 3,5-dinitrophenyl. Non-limiting examples of C₁-C₆ trihaloalkyl C₆-C₁₀ aryl groups include 2-trifluoromethylphenyl, 3-trifluoromethylphenyl, 4-trifluoromethylphenyl, 2,4-bis(trifluoromethyl)phenyl, 2,5-bis(trifluoromethyl)phenyl, 2,6-bis(trifluoromethyl)phenyl, and 3,5-bis(trifluoromethyl)phenyl. Non-limiting examples of hydroxy substituted C₆-C₁₀ aryl groups include 2-hydroxyphenyl, 3-hydroxyphenyl, 4-hydroxyphenyl, 2,4-dihydroxyphenyl, 2,5-dihydroxyphenyl, 2,6-dihydroxyphenyl, 3,5-dihydroxyphenyl, and 3,4,5-trihydroxyphenyl. Non-limiting examples of —OR⁵ substituted C₆-C₁₀ aryl groups include 2-methoxyphenyl, 3-methoxyphenyl, 4-methoxyphenyl, 2,4-dimethoxyphenyl, 2,5-dimethoxyphenyl, 2,6-dimethoxyphenyl, 3,5-dimethoxyphenyl, and 3,4,5-trimethoxyphenyl, as well as ethoxy, propoxy, isopropoxy, butoxy, sec-butoxy, isobutoxy, tert-butoxy, and prop-2-ynyloxy analogs thereof. A non-limiting examples of C₆-C₁₀ aryl group having combinations of substituents includes 3-bromo-4-fluorophenyl.

In certain embodiments, R¹ is a naphthyl group optionally substituted with halo, nitro, C₁-C₆ trihaloalkyl, hydroxyl, and —OR⁵ substituents. Non-limiting examples of substituted naphthyl groups include 4-chloro-1-naphthyl, 1-bromo-2-naphthyl, and 6-bromo-2-naphthyl.

In certain embodiments, R¹ is a C₆-C₁₀ aryl group substituted with a C₆-C₁₀ aryl group. Non-limiting examples of C₆-C₁₀ aryl groups substituted with a C₆-C₁₀ aryl group include 2-phenylphenyl, 3-phenylphenyl, and 4-phenylphenyl (i.e., 1,4-biphenyl).

In certain embodiments, R¹ is a heterocycloaryl group optionally substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, 3-cyano-1,2,5-oxadiazol-4-yl-2-oxide, C₁-C₆ haloalkyl, C₁-C₆ dihaloalkyl, C₁-C₆ trihaloalkyl, —NO₂, —OH, —OR⁴, —SH, —SR⁴, —COR⁴, —COOR⁴, —CONHR⁴, and —CONHR⁴R⁵. The heterocycloaryl group can be a monocyclic heterocycloaryl group or can be fused to a C₆-C₁₀ aryl group to form a bicyclic heterocycloaryl group. In preferred embodiments, R¹ is a heterocycloaryl group selected from the group consisting of furan-2-yl, thiophen-2-yl, 2-pyridyl, 3-pyridyl, and 4-pyridyl, wherein the heterocycloaryl group is optionally substituted as described herein. The heterocycloaryl group can be substituted at any open position on the heterocycloaryl group.

In certain embodiments, R¹ is methylenedioxyphenyl, 2,3-benzofuranyl, or 2,3-dihydrobenzofuranyl.

In an embodiment of Formula I, the invention provides a compound of Formula II:

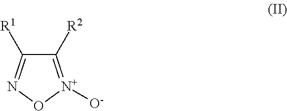

wherein R¹ is selected from the group consisting of a C₆-C₁₀ aryl group, a heterocycloaryl group, and R⁶, each optionally substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, C₆-C₁₀ heterocycloaryl, 3-cyano-1,2,5-oxadiazol-4-yl-2 oxide, C₁-C₆ haloalkyl, C₁-C₆ dihaloalkyl, C₁-C₆ trihaloalkyl, —NO₂, —OH, —OR⁴, —SH, —SR⁴, —SOR⁴, —SO₂R⁴, —COR⁴, —COOH, —COOR⁴, —CONHR⁴, and —CONHR⁴R⁵,

R² is selected from the group consisting of C₁-C₆ alkyl, C₂-C₆ alkenyl, C₂-C₆ alkynyl, C₃-C₈ cycloalkyl, C₃-C₈ cycloalkenyl, —CH₂OH, —CHO, —COOH, —CONH₂, —C═NR⁵, —C═NOH, —C═NOR⁵, and —CN,

R³, R⁴, and R⁵ are selected from the group consisting of C₁-C₆ alkyl, C₂-C₆ alkenyl, C₂-C₆ alkynyl, C₃-C₈ cycloalkyl, and C₃-C₈ cycloalkenyl, and

R⁶ is methylenedioxyphenyl, 2,3-benzofuranyl, or 2,3-dihydrobenzofuranyl.

In certain embodiments of Formula I, the invention provides a compound of Formula (IV):

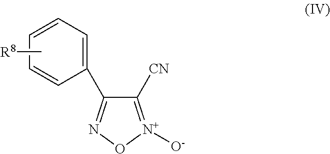

wherein R⁸ is one or more groups selected from the group consisting of halo, C₁-C₆ trihaloalkyl, —NO₂, —OH, and —OR⁵.

In certain embodiments of Formula I, the invention provides a compound of Formula (V):

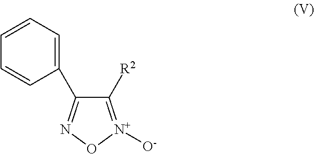

wherein R² is as defined herein.

In certain preferred embodiments, the invention provides a compound selected from the group consisting of 3-cyano-4-(4-fluorophenyl)-1,2,5-oxadiazole-2-oxide, 3-cyano-4-(4-chlorophenyl)-1,2,5-oxadiazole-2-oxide, 3-cyano-4-(4-bromophenyl)-1,2,5-oxadiazole-2-oxide, 3-cyano-4-(4-trifluoromethylphenyl)-1,2,5-oxadiazole-2-oxide, 3-cyano-4-(4-nitrophenyl)-1,2,5-oxadiazole-2-oxide, 3-cyano-4-(4-methoxyphenyl)-1,2,5-oxadiazole-2-oxide, 3-cyano-4-p-tolyl-1,2,5-oxadiazole-2-oxide, 3-cyano-4-(biphenyl-4-yl)-1,2,5-oxadiazole-2-oxide, 3-cyano-4-(4-(prop-2-ynyloxy)phenyl)-1,2,5-oxadiazole-2-oxide, 3-cyano-4-(3-nitrophenyl)-1,2,5-oxadiazole-2-oxide, 3-cyano-4-(3-chlorophenyl)-1,2,5-oxadiazole-2-oxide, 3-cyano-4-(3-bromophenyl)-1,2,5-oxadiazole-2-oxide, 3-cyano-4-(3-trifluoromethylphenyl)-1,2,5-oxadiazole-2-oxide, 3-cyano-4-(3-methoxyphenyl)-1,2,5-oxadiazole-2-oxide, 3-cyano-4-(3-hydroxyphenyl)-1,2,5-oxadiazole-2-oxide, 3-cyano-4-(2-methoxyphenyl)-1,2,5-oxadiazole-2-oxide, 3-cyano-4-(3-bromo-4-fluorophenyl)-1,2,5-oxadiazole-2-oxide, 3-cyano-4-(4-chloro-3-nitrophenyl)-1,2,5-oxadiazole-2-oxide, 3-cyano-4-(3,5-bis(trifluoromethylphenyl))-1,2,5-oxadiazole-2-oxide, 3-cyano-4-(3,4,5-trimethoxyphenyl)-1,2,5-oxadiazole-2-oxide, 3-cyano-4-(benzo[d][1,3]dioxol-5-yl)-1,2,5-oxadiazole-2-oxide, and 3-cyano-4-(naphthalene-2-yl)-1,2,5-oxadiazole-2-oxide.

In certain preferred embodiments, the invention provides a compound selected from the group consisting of 3-cyano-4-(4-fluorophenyl)-1,2,5-oxadiazole-2-oxide, 3-cyano-4-(4-chlorophenyl)-1,2,5-oxadiazole-2-oxide, 3-cyano-4-(4-bromophenyl)-1,2,5-oxadiazole-2-oxide, 3-cyano-4-(4-trifluoromethylphenyl)-1,2,5-oxadiazole-2-oxide, 3-cyano-4-(4-nitrophenyl)-1,2,5-oxadiazole-2-oxide, 3-cyano-4-(3-nitrophenyl)-1,2,5-oxadiazole-2-oxide, 3-cyano-4-(3-chlorophenyl)-1,2,5-oxadiazole-2-oxide, 3-cyano-4-(3-bromophenyl)-1,2,5-oxadiazole-2-oxide, 3-cyano-4-(3-trifluoromethylphenyl)-1,2,5-oxadiazole-2-oxide, 3-cyano-4-(3-bromo-4-fluorophenyl)-1,2,5-oxadiazole-2-oxide, 3-cyano-4-(4-chloro-3-nitrophenyl)-1,2,5-oxadiazole-2-oxide, and 3-cyano-4-(3,5-bis(trifluoromethylphenyl))-1,2,5-oxadiazole-2-oxide.

In a certain embodiment, the invention provides a compound of Formula III:

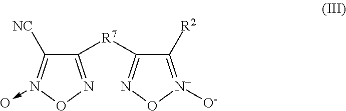

wherein R⁷ is selected from the group consisting of a C₆-C₁₀ aryl group and a heterocycloaryl group, and wherein each is optionally further substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, C₆-C₁₀ heterocycloaryl, C₁-C₆ haloalkyl, C₁-C₆ dihaloalkyl, C₁-C₆ trihaloalkyl, —NO₂, —OH, —OR⁴, —SH, —SR⁴, —SOR⁴, —SO₂R⁴, —COR⁴, —COOH, —COOR⁴, —CONHR⁴, and —CONHR⁴R⁵, and

R² is selected from the group consisting of C₁-C₆ alkyl, C₂-C₆ alkenyl, C₂-C₆ alkynyl, C₃-C₈ cycloalkyl, C₃-C₈ cycloalkenyl, —CH₂OH, —CHO, —COOH, —CONH₂, —C═NR⁵, —C═NOH, —C═NOR⁵, and —CN.

In certain embodiments, R⁷ in Formula III is optionally further substituted by 1, 2, 3, 4, or 5 substituents selected from the group consisting of halo, C₁-C₆ trihaloalkyl, nitro, hydroxy, and —OR⁴, and

R² is —CN.

In certain preferred embodiments, the invention provides a compound of Formula III selected from the group consisting of 4,4′-(1,3-phenylene)bis(3-cyano-1,2,5-oxadiazole-2-oxide), 4,4′-(1,4-phenylene)bis(3-cyano-1,2,5-oxadiazole-2-oxide), and 4,4′-(5-fluoro-1,3-phenylene)bis(3-cyano-1,2,5-oxadiazole-2-oxide).

In certain preferred embodiments, the invention provides a compound of Formula III that is selected from the group consisting of 4,4′-(thiophen-2,4-diyl)bis(3-cyano-1,2,5-oxadiazole 2-oxide) and 4,4′-(thiophen-2,5-diyl)bis(3-cyano-1,2,5-oxadiazole 2-oxide).

In accordance with an embodiment, A in Formula (I) is —C(═O)— (i.e., a carbonyl group). The carbonyl group is bonded to R¹ and to the 4-position of the 1,2,5-oxadiazole ring. In these embodiments, R¹ and R² are as defined previously herein.

In certain preferred embodiments, the invention provides a compound selected form the group consisting of 3-cyano-4-(furan-2-yl)-1,2,5-oxadiazole-2-oxide, 3-cyano-4-(5-nitrofuran-2-yl)-1,2,5-oxadiazole-2-oxide, and 3-cyano-4-(thiophen-2-yl)-1,2,5-oxadiazole-2-oxide. In a preferred embodiment, the invention provides 3-cyano-4-thienoyl-furoxan.

It will be understood that A is bonded to the 4-position of the oxadiazole ring, and that the group R² is bonded to the 3-position of the oxadiazole ring.

The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts. Preferred pharmaceutically acceptable salts of inventive compounds having a basic moiety (e.g., a pyridyl group) include hydrochloride and hydrobromide salts. The compounds of the present invention containing an acidic or basic moiety are useful in the form of the free base or acid or in the form of a pharmaceutically acceptable salt thereof.

It should be recognized that the particular counterion foaming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

Referring now to terminology used generically herein, the term “alkyl” means a straight-chain or branched alkyl substituent containing from, for example, 1 to about 6 carbon atoms, preferably from 1 to about 4 carbon atoms, more preferably from about 1 to about 2 carbon atoms. Examples of such substituents include methyl, ethyl, propyl, isopropyl, n-butyl, sec-butyl, isobutyl, tert-butyl, pentyl, isoamyl, hexyl, and the like.

The term “alkenyl,” as used herein, means a linear alkenyl substituent containing at least one carbon-carbon double bond and from, for example, about 2 to about 6 carbon atoms (branched alkenyls are about 3 to about 6 carbons atoms), preferably from about 2 to about 5 carbon atoms (branched alkenyls are preferably from about 3 to about 5 carbon atoms), more preferably from about 3 to about 4 carbon atoms. Examples of such substituents include propenyl, isopropenyl, n-butenyl, sec-butenyl, isobutenyl, tert-butenyl, pentenyl, isopentenyl, hexenyl, and the like.

The term “alkynyl,” as used herein, means a linear alkynyl substituent containing at least one carbon-carbon triple bond and from, for example, about 2 to about 6 carbon atoms (branched alkynyls are about 3 to about 6 carbons atoms), preferably from about 2 to about 5 carbon atoms (branched alkynyls are preferably from about 3 to about 5 carbon atoms), more preferably from about 3 to about 4 carbon atoms. Examples of such substituents include propynyl, isopropynyl, n-butynyl, sec-butynyl, isobutynyl, tert-butynyl, pentynyl, isopentynyl, hexynyl, and the like.

The term “cycloalkyl,” as used herein, means a cyclic alkyl substituent containing from, for example, about 3 to about 8 carbon atoms, preferably from about 4 to about 7 carbon atoms, and more preferably from about 4 to about 6 carbon atoms. Examples of such substituents include cyclopropyl, cyclobutyl, cyclopentyl, cyclohexyl, cycloheptyl, cyclooctyl, and the like. The term “cycloalkenyl,” as used herein, means the same as the term “cycloalkyl,” however one or more double bonds are present. Examples of such substituents include cyclopentenyl and cyclohexenyl. The cyclic alkyl groups may be unsubstituted or further substituted with alkyl groups such as methyl groups, ethyl groups, and the like.

The term “heterocycloaryl,” as used herein, refers to a monocyclic or bicyclic 5- or 6-membered aromatic ring system containing one or more heteroatoms selected from the group consisting of O, N, S, and combinations thereof. Examples of suitable monocyclic heterocycloaryl groups include but are not limited to furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, pyridinyl, pyrimidinyl, and triazinyl. The heterocycloaryl group can be attached to the 1,2,5-oxadiazole ring at any available position on the heterocycloaryl group. For example, a furanyl group can be attached at the 2-position or the 3-position of the furanyl group. A pyridyl group can be attached at the 2-, 3-, or 4-position of the pyridyl group. Suitable bicyclic heterocycloaryl groups include monocylic heterocycloaryl rings fused to a C₆-C₁₀ aryl ring. Non-limiting examples of bicyclic heterocycloaryl groups include benzofuran, benzothiophene, quinoline, and isoquinoline. The heterocycloaryl group is optionally substituted with 1, 2, 3, 4, or 5 substituents as recited herein, wherein the optional substituent can be present at any open position on the heterocycloaryl group.

The term “halo” or “halogen,” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2 π electrons, according to Hückel's Rule.

The term “arylfuroxan” is synonymous with the term “1,2,5-oxadiazole-2-oxide.” Thus, the term “4-aryl-3-cyanofuroxan” refers to 4-aryl-3-cyano-1,2,5-oxadiazole-2-oxide.

Some abbreviations used herein are defined as follows: THF, tetrahydrofuran; DMF, dimethylformamide; and DMSO, dimethyl sulfoxide.

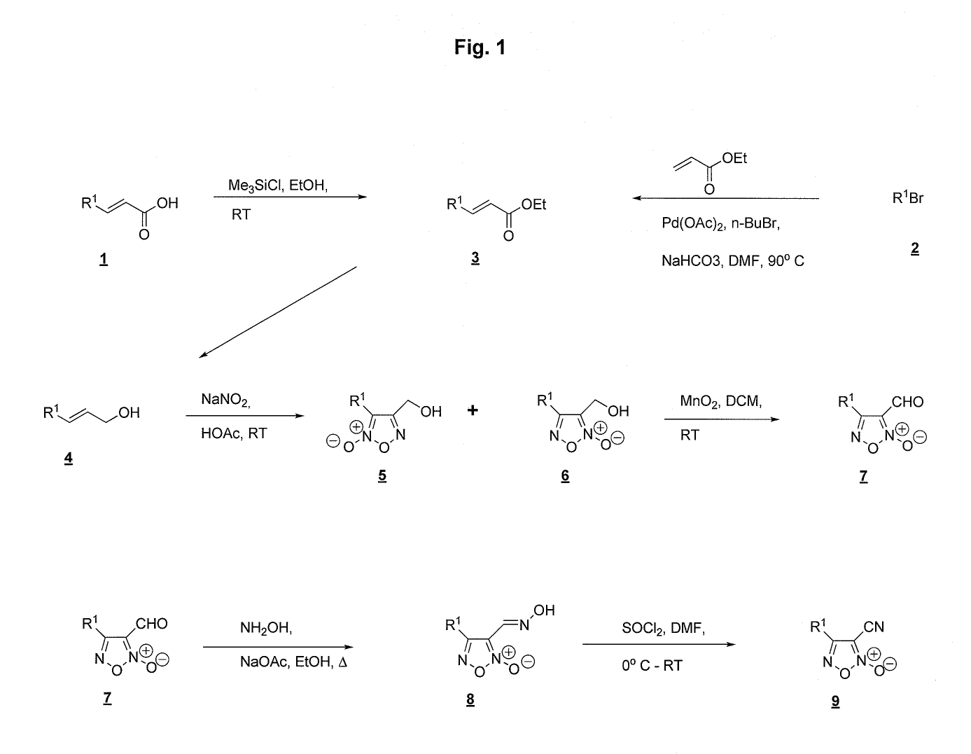

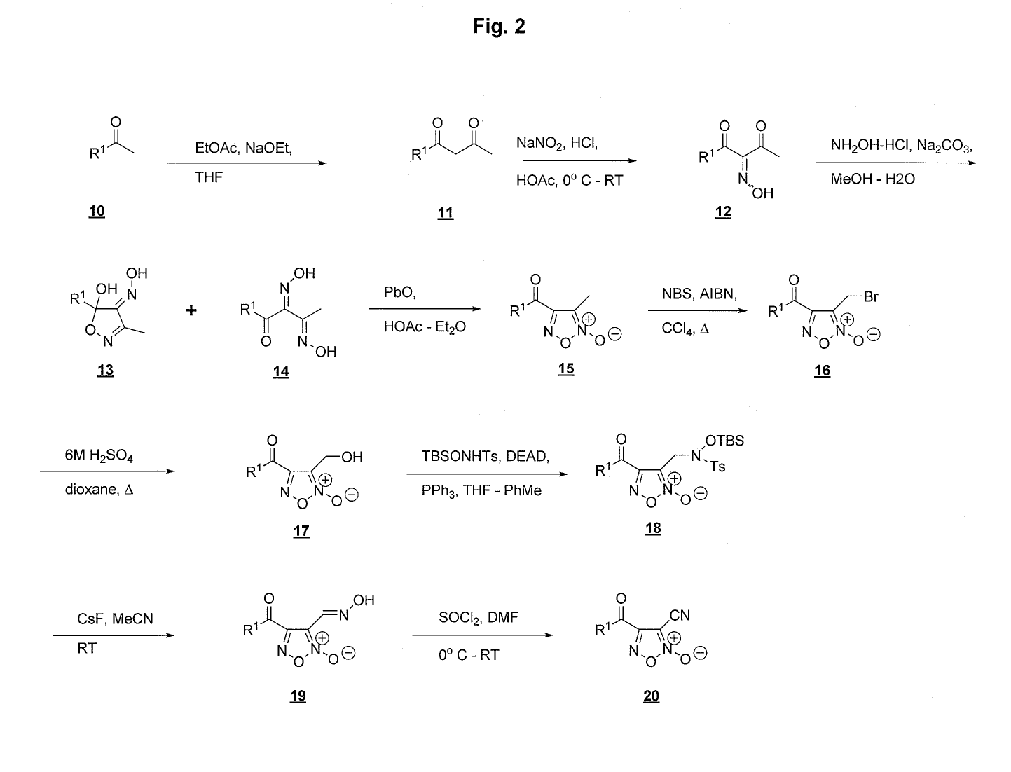

The compounds of the invention can be synthesized according to the procedures set forth in FIGS. 1 and 2, wherein R¹ is as defined herein.

FIG. 1 shows a method of preparation of compounds defined by Formula I, wherein A is a bond.

The reaction of 3-arylated 2-propenoic acid 1 with alcohols R′OH in the presence of trialkylsilyl reagents such as chlorotrimethylsilane gives 3-arylated 2-propenoic esters 3. Alternatively, 3-arylated 2-propenoic esters 3 are prepared by Heck coupling of aryl or heterocycloaryl halides 2 with acrylic acid esters in the presence of a Pd catalyst such as Pd(OAc)₂, and a base such as sodium carbonate in a suitable solvent such as dimethylformamide.

Reduction of 3-arylated 2-propenoic esters 3 with reducing agents such as diisobutylaluminum hydride (“DIBAL”) in a solvent such as toluene, THF, or mixtures thereof gives allylic alcohols 4. Cyclization of allylic alcohols 4 with sodium nitrite in acetic acid at room temperature gives 4-aryl-3-hydroxymethyl-1,2,5-oxadiazolyl-2-oxides 5 and 6, with isomer 6 being formed in major amount. Separation of desired isomer 6 from minor isomer 5 can be effected by chromatography or crystallization.

Oxidation of 4-aryl-3-hydroxymethyl-1,2,5-oxadiazolyl-2-oxide 6 with oxidizing agents such as MnO₂ in a solvent such as dichloromethane provides aldehyde 7. Treatment of aldehyde 7 with hydroxylamine hydrochloride in the presence of a base such as sodium acetate in a solvent such as ethanol gives oxime 8. Dehydration of oxime 8 with thionyl chloride-DMF gives the 4-aryl-3-cyano-1,2,5-oxadiazolyl-2-oxides 9.

FIG. 2 shows a method of preparation of compounds defined by Formula I, wherein A is —C(═O)— (i.e., a carbonyl group).

Reaction of aryl ketone or heterocycloaryl ketone 10 with an ester such as ethyl acetate in the presence of a base such as sodium ethoxide in a solvent such as THF gives the β-diketone 11. Treatment of β-diketone 11 with nitrous acid generated from sodium nitrite and an acid such as hydrochloric acid in a solvent such as acetic acid gives the diketooxime 12. Treatment of diketooxime 12 with hydroxylamine hydrochloride in the presence of a base such as sodium carbonate in a solvent such as MeOH—H₂O gives the bis oxime 14, accompanied by a minor amount of the cyclized compound 13.

Cyclization of the bis oxime 14 by treatment with an oxidizing agent such as PbO in a solvent such as HOAc-Et₂O gives 4-aryl-3-methyl-1,2,5-oxadiazol-2-yl oxide 15. Halogenation of the methyl group of compound 15 with a halogenating agent such as N-bromosuccinimide in the presence of a radical initiator such as azobisisobutyronitrile (“AIBN”) in a solvent such as CCl₄ gives the bromomethyl compound 16. Solvolysis of bromomethyl compound 16 in an aqueous medium such as 6M H₂SO₄ and dioxane gives hydroxymethyl compound 17. Mitsonobu reaction of hydroxymethyl compound 17 with N-(tert-butyldimethylsilyloxy)benzenesulfonamide to give compound 18 followed by treatment with cesium fluoride in a solvent such as acetonitrile gives oxime 19. Dehydration of oxime 19 with thionyl chloride-DMF gives the 4-aryl-3-cyano-1,2,5-oxadiazolyl-2-oxides 20.

The present invention is further directed to a pharmaceutical composition comprising a pharmaceutically acceptable carrier and at least one compound selected from the group consisting of the presently described compounds.

The pharmaceutically acceptable excipients described herein, for example, vehicles, adjuvants, carriers or diluents, are well-known to those who are skilled in the art and are readily available to the public. It is preferred that the pharmaceutically acceptable carrier be one that is chemically inert to the active compounds and one that has no detrimental side effects or toxicity under the conditions of use.

The choice of excipient will be determined in part by the particular compound of the present invention chosen, as well as by the particular method used to administer the composition. Accordingly, there is a wide variety of suitable formulations of the pharmaceutical composition of the present invention. The following formulations for oral, aerosol, parenteral, subcutaneous, intravenous, intramuscular, intraperitoneal, intrathecal, rectal, and vaginal administration are merely exemplary and are in no way limiting.

One skilled in the art will appreciate that suitable methods of utilizing a compound and administering it to a mammal for the treatment of disease states, in particular, schistosomiasis, which would be useful in the method of the present invention, are available. Although more than one route can be used to administer a particular compound, a particular route can provide a more immediate and more effective reaction than another route. Accordingly, the described methods are merely exemplary and are in no way limiting.

The dose administered to an animal, particularly human and other mammals, in accordance with the present invention should be sufficient to effect the desired response. Such responses include reversal or prevention of the bad effects of the disease, in particular, schistosomiasis, for which treatment is desired or to elicit the desired benefit. One skilled in the art will recognize that dosage will depend upon a variety of factors, including the age, species, condition or disease state, and body weight of the animal, as well as the source and extent of the disease condition in the animal. The size of the dose will also be determined by the route, timing and frequency of administration as well as the existence, nature, and extent of any adverse side-effects that might accompany the administration of a particular compound and the desired physiological effect. It will be appreciated by one of skill in the art that various conditions or disease states may require prolonged treatment involving multiple administrations.

Suitable doses and dosage regimens can be determined by conventional range-finding techniques known to those of ordinary skill in the art. Generally, treatment is initiated with smaller dosages that are less than the optimum dose of the compound. Thereafter, the dosage is increased by small increments until the optimum effect under the circumstances is reached. The present inventive method typically will involve the administration of about 0.1 to about 300 mg of one or more of the compounds described above per kg body weight of the individual.

The invention further provides a method for treating schistosomiasis in a mammal comprising administering an effective amount of the compound of the invention to a mammal afflicted therewith. Desirably, schistosomiasis can be treated at any stage in the life cycle of schistosomiasis parasites such as S. mansoni.

Adult S. mansoni parasites live in an aerobic environment within human hosts, and therefore must have effective mechanisms to maintain cellular redox balance. Additionally, worms must be able to evade reactive oxygen species generated by the host's immune response. In most eukaryotes there are two major systems to detoxify reactive oxygen species, one based on the tripeptide glutathione and the other based on the protein thioredoxin. Glutathione reductase (GR) reduces glutathione disulfide, whereas thioredoxin reductases (TrxR) are pivotal in the Trx-dependent system.

It was recently discovered that in S. mansoni, specialized TrxR and GR enzymes are absent, and instead are replaced by a unique multifunctional enzyme, thioredoxin glutathione reductase (TGR) (see, e.g., Alger et al., Mol. Biochem. Parasitol. 121, 129-139 (2002). This reliance on a single enzyme for both glutathione disulfide and thioredoxin reduction suggests that the parasite's redox systems are subject to a bottleneck dependence on TGR, and that TGR represents a potentially important drug target.

The invention further provides a use of a compound of the invention in the manufacture of a medicament for treating schistosomiasis. The medicament typically is a pharmaceutical composition as described herein.

The invention additionally provides a method of inhibiting thioredoxin glutathione reductase (“TGR”) of S. mansoni in a mammal invaded by S. mansoni. The method comprises administering a compound or salt of the invention to the mammal. Preferably, the mammal is a human.

The following examples further illustrate the invention but, of course, should not be construed as in any way limiting its scope.

### Example 1

Unless otherwise stated, all reactions were carried out under an atmosphere of dry argon or nitrogen in dried glassware. Indicated reaction temperatures refer to those of the reaction bath, while room temperature (rt) is noted as 25° C. All solvents were of anhydrous quality purchased from Aldrich Chemical Co. and used as received. Commercially available starting materials and reagents were purchased from Aldrich, TCI and Acros and were used as received.

Analytical thin layer chromatography (TLC) was performed with Sigma Aldrich TLC plates Aldrich TLC plates (5×20 cm, 60 Å, 250 μm). Visualization was accomplished by irradiation under a 254 nm UV lamp. Chromatography on silica gel was performed using forced flow (liquid) of the indicated solvent system on Biotage KP-Sil pre-packed cartridges and using the Biotage SP-1 automated chromatography system. ¹H- and ¹³C NMR spectra were recorded on a Varian Inova 400 MHz spectrometer. Chemical shifts are reported in ppm with the solvent resonance as the internal standard (CDCl₃ 7.26 ppm, 77.00 ppm, DMSO-d₆ 2.5 ppm, 39.51 ppm for ¹H, ¹³C respectively). Data are reported as follows: chemical shift, multiplicity (s=singlet, d=doublet, dd=doublet of doublet, t=triplet, q=quartet, br=broad, m=multiplet), coupling constants, and number of protons. Low resolution mass spectra (electrospray ionization) were acquired on an Agilent Technologies 6130 quadrupole spectrometer coupled to an Agilent Technologies 1200 series HPLC. High resolution mass spectral data was collected in-house using and Agilent 6210 time-of-flight mass spectrometer, also coupled to an Agilent Technologies 1200 series HPLC system.

This example demonstrates a procedure for preparing an intermediate leading to compounds in accordance with an embodiment, specifically, the esterification of cinnamic acids to provide the corresponding cinnamic acid esters.

To a solution containing a substituted cinnamic acid (10 mmol) in absolute ethanol (50 mL) was added TMSCl (22 mmol) and the reaction was stirred at RT for 12 h. After completion of the reaction, the solvent was removed under diminished pressure and the residue was re-dissolved in ethyl acetate. The ethyl acetate layer was washed successively with saturated NaHCO₃, water, brine then dried (Na₂SO₄) and concentrated under diminished pressure to give the pure product without need of further purification.

### Example 2

This example demonstrates a procedure for the preparation of cinnamic acid esters via Heck coupling of aryl bromides with alkyl acrylates.

To a solution containing an aryl bromide (10 mmol) in DMF (20 mL) was added tetrabutylammonium bromide (10 mmol), palladium(II) acetate (5 mol %), sodium bicarbonate (40 mmol), and ethyl acrylate (20 mmol). The reaction mixture was stirred at 90° C. for 45 min, then cooled to room temperature, diluted with ethyl acetate and filtered through celite. The filtrate was poured into water and extracted with ethyl acetate. The organic layer was washed with water and brine then dried (MgSO₄), filtered and concentrated under diminished pressure. The crude solid was purified on a Biotage™ silica gel column. Gradient elution with ethyl acetate (7→40%) in hexanes gave the product.

### Example 3

This examples demonstrates a procedure for the preparation of cinnamic alcohols from the corresponding cinnamic acid esters.

To a suspension of the corresponding ester (10 mmol) in toluene (25 mL) at −78° C. was added dropwise DIBAL (22 mmol, 1.0 M solution in toluene) over 45 min. The reaction mixture was allowed to warm to room temperature over 2 h and then stirred this temperature for an additional hour. The reaction mixture was quenched with ice containing dilute HCl. The organic layer was separated and the aqueous layer was extracted with ethyl acetate. The combined organic layer was washed with brine and water, dried (MgSO₄), filtered and concentrated under diminished pressure. The crude residue was purified on a Biotage™ silica gel column. Gradient elution with ethyl acetate (12→80%) in hexanes gave the product.

### Example 4

This example demonstrates a procedure for the preparation of 4-arylfuroxan-3-methanol compounds from the appropriate cinnamyl alcohols.

To a solution of cinnamyl alcohol (10 mmol) in glacial acetic acid (50 mL) was added sodium nitrite (10-20 mmol for deactivated aryl groups/4 mmol for activated aryl groups) portion wise over 45 min. The reaction mixture was stirred at RT for 6-48 h. After completion of the reaction, the reaction mixture was quenched with ice water and extracted with ethyl acetate. The ethyl acetate layer was washed successively with saturated NaHCO₃, water, brine then dried (Na₂SO₄) and concentrated under diminished pressure to give the crude product. The crude residue was purified on a Biotage™ silica gel column. Gradient elution with ethyl acetate (7→80%) in hexanes gave the product containing a small amount of the isomeric 1,2,5-oxadiazole-2-oxide (compounds 6 and 5, respectively, of FIG. 1).

### Example 5

This example demonstrates a procedure for the preparation of 4-arylfuroxan-3-carboxaldehydes from the appropriate cinnamyl alcohols.

4-Aryfuroxan-3-methanol (10 mmol) was dissolved in dichloromethane (50 mL) and treated with activated manganese dioxide (150 mmol). The reaction mixture was stirred at RT for 6-12 h then filtered through celite and concentrated under diminished pressure. The crude product was purified on a Biotage™ silica gel column. Gradient elution with dichloromethane (12→100%) in hexanes gave the product.

### Example 6

This example demonstrates a procedure for the preparation of 4-arylfuroxan-3-caroxaldehyde oximes from the corresponding 4-arylfuroxan-3-carobxaldehydes.

4-Aryfuroxan-3-methanal (10 mmol), hydroxylamine hydrochloride (15 mmol) and sodium acetate (10 mmol) in ethanol (50 mL) was refluxed for 10-20 min. After completion of the reaction, the solvent was removed under diminished pressure and the crude residue was purified on a Biotage™ silica gel column. Gradient elution with ethyl acetate (7→60%) in hexanes gave the product.

### Example 7

This example demonstrates a procedure for the preparation of 4-aryl-cyanofuroxans from the corresponding 4-arylfuroxan-3-carboxaldehyde oximes.

To a solution of 4-arylfuroxan-3-carbaldehyde oxime (1 mmol) in DMF (4 mL) was added drop wise thionyl chloride (4 mmol) at 0° C. The reaction mixture was allowed to warm to room temperature with stirring over 3 h then stirred at this temperature for an additional hour. The reaction mixture was quenched with ice and extracted with dichloromethane. The combined organic layer was successively washed with saturated NaHCO₃, water, brine then dried (Na₂SO₄) and concentrated under diminished pressure to give the crude product. The crude product was purified on a Biotage™ silica gel column/preparative HPLC. Gradient elution with ethyl acetate (1→25%) in hexanes gave the product.

The following compounds were prepared by the method illustrated in Examples 1-7 and illustrated schematically in FIG. 1

### 3-cyano-4-(4-fluorophenyl)-1,2,5-oxadiazole-2-oxide (21)

¹H NMR (DMSO-d₆) δ 7.53-7.58 (m, 2H, Ar—H) and 7.92-7.96 (m, 2H, Ar—H); ¹³C-NMR (DMSO-d₆); δ98.4, 107.3, 117.0 (d, J=22.3 Hz), 120.5 (d, J=3.0 Hz), 129.8 (d, J=8.9 Hz), 154.4 and 164.4 (d, J=250.7 Hz).

### 4-(4-chlorophenyl)-3-cyano-1,2,5-oxadiazole-2-oxide (22)

¹H NMR (DMSO-d₆) δ 7.75 (d, J=8.4 Hz, 2H, Ar—H) and 7.86 (d, J=8.8 Hz, 2H, Ar—H); ¹³C NMR (DMSO-d₆); δ 99.1, 108.0, 123.5, 128.7, 129.8, 130.4, 131.5, 138.1 and 155.0.

### 4-(4-bromophenyl)-3-cyano-1,2,5-oxadiazole-2-oxide (23)

¹H NMR (DMSO-d₆) δ 7.77 (d, J=8.4 Hz, 2H, Ar—H) and 7.90 (d, J=8.4 Hz, 2H, Ar—H); ¹³C NMR (DMSO-d₆); δ 99.0, 108.0, 123.9, 127.0, 128.8, 130.5, 132.7, 134.4 and 155.0.

### 3-cyano-4-(4-(trifluoromethyl)phenyl)-1,2,5-oxadiazole-2-oxide (24)

¹H NMR (CDCl₃) δ 7.87 (d, J=8.8 Hz, 2H, Ar—H) and 8.08 (d, J=8.8 Hz, 2H, Ar—H); ¹³C NMR (CDCl₃); δ 95.3, 106.1, 120.5, 125.9 (q, J_{C-F}=271.5 Hz), 126.7, 126.8 (q, J_{C-F}=3.7 Hz), 127.2, 127.4, 134.2, 134.8 (q, J_{C-F}=32.8 Hz) and 153.1.

### 3-cyano-4-(4-nitrophenyl)-1,2,5-oxadiazole-2-oxide (25)

¹H NMR (CDCl₃) δ 8.16 (d, J=8.4 Hz, 2H, Ar—H) and 8.46 (d, J=8.4 Hz, 2H, Ar—H); ¹³C NMR (CDCl₃) δ 94.5, 105.9, 124.3, 124.9, 128.1, 129.5, 133.5, 150.1 and 152.4.

### 3-cyano-4-(4-methoxyphenyl)-1,2,5-oxadiazole-2-oxide (26)

¹H-NMR (CDCl₃) δ 3.91 (s, 3H, OCH₃), 7.06 (d, J=8.8 Hz, 2H, Ar—H) and 7.87 (d, J=9.2 Hz, 2H, Ar—H); ¹³C-NMR (CDCl₃) δ 55.6, 95.5, 106.7, 115.1, 115.9, 128.2, 128.5, 153.9 and 163.0.

### 3-cyano-4-p-tolyl-1,2,5-oxadiazole-2-oxide (27)

¹H NMR (CDCl₃) δ 2.47 (s, 3H, CH₃), 7.38 (d, J=8.0 Hz, 2H, Ar—H) and 7.81 (d, J=8.4 Hz, 2H, Ar—H); ¹³C NMR (CDCl₃) δ 22.0, 95.8, 106.8, 121.0, 127.0, 131.0, 146.0 and 154.8.

### 4-(biphenyl-4-yl)-3-cyano-1,2,5-oxadiazole-2-oxide (28)

¹H NMR (CDCl₃) δ 7.43-7.66 (m, 5H, Ar—H), 7.81 (d, J=8.8 Hz, 2H, Ar—H) and 8.01 (d, J=8.8 Hz, 2H, Ar—H); ¹³C NMR (CDCl₃) δ 95.7, 106.4, 122.2, 127.1, 127.2, 128.1, 128.3, 129.1, 138.2, 146.0 and 154.0.

### 3-cyano-4-(3-nitrophenyl)-1,2,5-oxadiazole-2-oxide (29)

¹H NMR (CDCl₃) δ 7.85 (t, J=8.0 Hz, 1H, Ar—H), 8.24-8.52 (m, 2H, Ar—H) and 8.84 (t, J=1.8 Hz, 1H, Ar—H); ¹³C NMR (CDCl₃) δ 94.2, 106.0, 122.0, 125.5, 127.1, 131.0, 132.2 149.0 and 152.3.

### 4-(3-chlorophenyl)-3-cyano-1,2,5-oxadiazole-2-oxide (30)

¹H NMR (DMSO-d₆) δ 7.34 (t, J=7.6 Hz, 1H, Ar—H), 7.79-7.85 (m, 2H, Ar—H) and 7.89 (t, J=1.6 Hz, 1H, Ar—H); ¹³C NMR (DMSO-d₆) δ 98.5, 107.2, 125.8, 125.9, 124.6, 131.8, 132.5, 134.2 and 154.1.

### 4-(3-bromophenyl)-3-cyano-1,2,5-oxadiazole-2-oxide (31)

¹H NMR (CDCl₃) δ 7.47 (t, J=8.0 Hz, 1H, Ar—H), 7.76-7.86 (m, 2H, Ar—H) and 8.09 (t, J=2 Hz, 1H, Ar—H); ¹³C NMR (CDCl₃) δ 95.3, 106.1, 119.2, 124.0, 126.0, 130.0, 131.2, 136.0 and 153.0.

### 3-cyano-4-(3-(trifluoromethyl)phenyl)-1,2,5-oxadiazole-2-oxide (32)

¹H NMR (CDCl₃) δ 7.72-8.13 (m, 3H, Ar—H) and 8.21 (s, 1H, Ar—H); ¹³C NMR (CDCl₃) δ 95.3, 106.0, 120.5, 125.8 (q, J_{C-F}=271.2 Hz), 123.7, 123.9 (q, J_{C-F}=4.1 Hz), 129.3, 129.4 (q, J_{C-F}=3.4 Hz), 130.0, 130.5, 132.4, 132.9 (q, J_{C-F}=33.5 Hz) and 153.1.

### 3-cyano-4-(3-methoxyphenyl)-1,2,5-oxadiazole-2-oxide (33)

¹H NMR (CDCl₃) δ 3.88 (s, 3H, OCH₃) and 7.14-7.50 (m, 4H, Ar—H); ¹³C NMR (CDCl₃) δ 55.53, 96.0, 106.4, 111.6, 116.8, 119.2, 124.9, 130.9 154.2 and 160.34.

### 3-cyano-4-(2-methoxyphenyl)-1,2,5-oxadiazole-2-oxide (34)

¹H NMR (CDCl₃) δ 3.96 (s, 3H, OCH₃), 7.06-7.15 (m, 2H, Ar—H) and 7.58-7.72 (m, 2H, Ar—H); ¹³C NMR (CDCl₃) δ 55.1, 98.4, 106.5, 111.6, 112.9, 121.4, 129.9, 134.3, 153.3 and 157.1.

### 4-(3-bromo-4-fluorophenyl)-3-cyano-1,2,5-oxadiazole-2-oxide (35)

¹H NMR (DMSO-d₆) δ 7.73 (t, J=8.6 Hz, 1H, Ar—H), 7.91-7.95 (m, 1H, Ar—H) and 8.18 (dd, J=6.4 and 2.0 Hz, 1H, Ar—H); ¹³C NMR (DMSO-d₆) δ 98.5, 107.1, 109.6 (d, J=22.3 Hz), 118.3 (d, J=23.1 Hz), 122.1, 129.1 (d, J=8.9 Hz), 132.3, 153.4 and 160.6 (d, J=250.8 Hz).

### 4-(4-chloro-3-nitrophenyl)-3-cyano-1,2,5-oxadiazole-2-oxide (36)

¹H NMR (CDCl₃) δ 7.83 (d, J=8.4 Hz, 1H, Ar—H), 8.07 (dd, J=8.4 and 2.4 Hz, 1H, Ar—H) and 8.48 (d, J=2 Hz, 1H, Ar—H); ¹³C NMR (CDCl₃); δ 94.9, 105.7, 123.9, 123.9, 130.5, 131.8, 133.6, 148.7 and 151.4.

### 4-(3,5-bis(trifluoromethyl)phenyl)-3-cyano-1,2,5-oxadiazole-2-oxide (37)

¹H NMR (CDCl₃) δ 8.16 (d, J=0.4 Hz, 2H, Ar—H) and 8.39 (d, J=0.4 Hz, 1H, Ar—H); ¹³C NMR (CDCl₃) δ 95.0, 105.6, 119.7, 125.1 (q, J_{C-F}=272.3 Hz), 126.1, 126.2-126.4 (m, C-F coupling)), 126.9, 127.0 (q, J_{C-F}=3 Hz), 133.3, 134.1 (q, J_{C-F}=34.5 Hz) and 151.8.

### 3-cyano-4-(3,4,5-trimethoxyphenyl)-1,2,5-oxadiazole-2-oxide (38)

¹H NMR (DMSO-d₆) δ 3.74 (s, 3H, OCH₃), 3.82 (s, 6H, OCH₃) and 7.11 (s, 2H, Ar—H); ¹³C NMR (DMSO-d₆) δ 56.1, 58.6, 61.7, 99.1, 104.5, 106.1, 108.2, 119.6, 141.4, 141.5, 154.3 and 155.6.

### 4-(benzo[d][1,3]dioxol-5-yl)-3-cyano-1,2,5-oxadiazole-2-oxide (39)

¹H NMR (DMSO-d₆) δ 6.19 (s, 2H, —OCH₂O—), 7.21 (d, J=8.0 Hz, 1H, Ar—H) and 7.36-7.4 (m, 2H, Ar—H); ¹³C NMR (DMSO-d₆) δ 98.3, 102.4, 106.4, 107.5, 109.3, 117.3, 122.4, 148.4, 150.9 and 154.7.

### 3-cyano-4-(naphthalen-2-yl)-1,2,5-oxadiazole-2-oxide (40)

¹H NMR (CDCl₃) 7.62-8.02 (m, 6H, Ar—H) and 8.42-8.43 (m, 1H, Ar—H); ¹³C NMR (CDCl₃) δ 95.8, 106.5, 121.0, 122.1, 127.5, 127.8, 128.0, 128.8, 129.0, 130.0 132.8, 135.0 and 154.5.

### 3-cyano-4-(furan-2-yl)-1,2,5-oxadiazole-2-oxide (41)

¹H NMR (DMSO-d₆) δ 6.88 (dd, J=3.8 Hz and 1.8 Hz, 1H, Het-H), 7.37 (dd, J=3.6 Hz and 0.8 Hz, 1H, Het-H), 8.18 (dd, J=1.8 Hz and 0.6 Hz, 1H, Het-H); ¹³C-NMR (DMSO-d₆) δ 96.8, 106.8, 113.0, 115.1, 138.2, 147.1 and 147.6.

### 3-cyano-4-(5-nitrofuran-2-yl)-1,2,5-oxadiazole-2-oxide (42)

¹H NMR (CDCl₃) δ 7.41 (d, J=4.0 Hz, 2H, Het-H) and 7.51 (d, J=4.0 Hz, 2H, Het-H); ¹³C NMR (CDCl₃) δ 93.8, 104.6, 112.0, 116.2, 139.3, 144.7 and 153.4.

### 4,4′-(1,3-phenylene)bis(3-cyano-1,2,5-oxadiazole-2-oxide) (43)

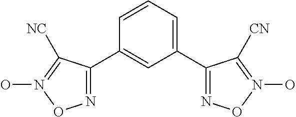

¹H NMR (CDCl₃) δ 7.87 (t, J=7.8 Hz, 1H, Ar—H), 8.19 (dd, J=7.8 and 1.8 Hz, 2H, Ar—H), 8.53-8.54 (m, 1H, Ar—H); ¹³C NMR (CDCl₃) δ 95.5, 106.0, 124.5, 125.8, 130.6, 131.2 and 153.0.

### 4,4′-(1,4-phenylene)bis(3-cyano-1,2,5-oxadiazole-2-oxide) (44)

¹H NMR (CDCl₃) δ 8.19 (s, 4H, Ar—H); ¹³C NMR (CDCl₃) δ 95.2, 106.1, 127.8, 128.1 and 152.8.

### 4,4′-(5-fluoro-1,3-phenylene)bis(3-cyano-1,2,5-oxadiazole-2-oxide) (45)

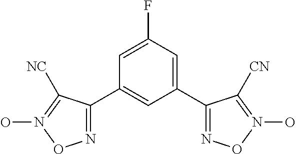

¹H NMR (CDCl₃) δ 7.92 (dd, J=8 Hz and 1.6 Hz, 2H, Ar—H) and 8.31-8.32 (m, 1H, Ar—H); ¹³C NMR (CDCl₃) δ 95.0, 105.7, 118.1 (d, J_{C-F}=23.8 Hz), 120.9 (d, J_{C-F}=3.7 Hz), 127.7 (d, J_{C-F}=8.2 Hz), 151.7 (d, J_{C-F}=2.2 Hz) and 163.3 (d, J_{C-F}=253 Hz).

### Example 8

This example illustrates a method of preparing 3-cyano-4-(3-hydroxyphenyl)-1,2,5-oxadiazole-2-oxide (46) and 3-cyano-4-(4-hydroxyphenyl)-1,2,5-oxadiazole-2-oxide (48) in accordance with an embodiment of the invention.

To a solution of 3-cyano-4-(3-methoxyphenyl)-1,2,5-oxadiazole-2-oxide or 3-cyano-4-(4-methoxyphenyl)-1,2,5-oxadiazole-2-oxide (1 mmol) in dichloromethane (5 mL) was treated with AlCl₃ (5 mmol) at 0° C. The reaction mixture was stirred for 24 h at 60° C. in a sealed tube. The reaction mixture was quenched with 1 N HCl and extracted with diethyl ether. The ether layer was washed successively with saturated NaHCO₃, water, brine then dried (Na₂SO₄) and concentrated under diminished pressure to give the crude product. The crude residue was purified on a Biotage™ silica gel column. Gradient elution with ethyl acetate (10→80%) in hexanes gave the product.

### 3-cyano-4-(3-hydroxyphenyl)-1,2,5-oxadiazole-2-oxide (46)

¹H NMR (DMSO-d₆) δ 7.06-7.30 (m, 3H, Ar—H), 7.70 (t, J=8.0 Hz, 1H, Ar—H) and 10.15 (s, 1H, OH); ¹³C-NMR (DMSO-d₆) δ 98.3, 107.5, 113.3, 117.5, 119.6, 124.9, 131.0, 155.0, 158.0.

### Example 9

This example illustrates a method of preparing 3-cyano-4-(4-(prop-2-ynyloxy)phenyl)-1,2,5-oxadiazole-2-oxide (47) in accordance with an embodiment of the invention.

To a solution of 3-cyano-4-(4-hydroxyphenyl)-1,2,5-oxadiazole-2-oxide (0.020 g, 0.098 mmol) in DMF (1 mL) was added cesium carbonate (0.048 g, 0.148 mmol) and 3-bromoprop-1-yne (0.018 g, 0.148 mmol) and stirred for 2 h at rt. After completion of the reaction, the reaction mixture was filtered and extracted with ethyl acetate. The organic layer was dried (Na₂SO₄) and solvent was removed. The crude product was purified in HPLC to afford a white solid: yield 0.018 g (0.073 mmol, 74%). ¹H NMR (CDCl₃) δ 2.58 (t, J=2.8 Hz, 1H, CH), 4.79 (d, J=2.4 Hz, 2H, CH₂), 7.16 (d, J=8.8 Hz, 2H, Ar—H) and 7.90 (d, J=9.2 Hz, 2H, Ar—H); ¹³C NMR (CDCl₃) δ 56.0, 76.5, 77.4, 95.5, 106.6, 116.0, 116.8, 128.5, 153.8 and 160.8.

### Example 10

This example illustrates a method of preparing 1-(thiophen-2-yl)butane-1,3-dione (11; R¹=2-thiophen-2-yl) in accordance with an embodiment of the invention.

To a solution containing 2-acetyl thiophene (10; R¹=2-thiophen-2-yl) (18 mL, 166 mmol) in THF (333 mL) was added sodium ethoxide (22.65 g, 333 mmol). The reaction mixture was stirred for 1 h at room temperature and then ethyl acetate (18 mL, 183 mmol) was added. The reaction mixture was then stirred for 6 h at room temperature and 1 h at reflux. The excess solvent was evaporated under diminished pressure then the residue was poured into ice water containing dilute HCl. The acidic solution was extracted with ethyl acetate and washed with brine, dried (MgSO₄), filtered and concentrated under diminished pressure. The crude residue was purified on a 340 g Biotage™ silica gel column. Gradient elution with ethyl acetate (6→40%) in hexanes gave the product 11 as a yellow solid: yield 21.8 g (130 mmol, 78%). The compound was characterized by LCMS and compared to the NMR data in the literature.¹

### Example 11

This example illustrates a method of preparing 2-(hydroxyimino)-1-(thiophen-2-yl)butane-1,3-dione (12; R¹=2-thiophen-2-yl) in accordance with an embodiment of the invention.

To a solution containing 1-(thiophen-2-yl)butane-1,3-dione (11) (4 g, 23.78 mmol) and concentrated HCl (3.61 mL, 119 mmol) in acetic acid (50 mL) at 0° C. was added dropwise sodium nitrite (2.46 g, 35.7 mmol) in 5 mL of water. The reaction mixture was stirred for 4 h at RT and then poured into ice water. The product was extracted with ethyl acetate. The organic layer was successively washed with water, saturated NaHCO₃, brine, dried (MgSO₄), filtered and concentrated under diminished pressure. The crude residue was purified on a 340 g Biotage™ silica gel column. Gradient elution with ethyl acetate (12→80%) in hexanes gave the product 12 as white crystals: yield 3.35 g (16.9 mmol, 71%). ¹H NMR (DMSO-d₆) δ 2.45 (s, 3H, CH₃), 7.25 (dd, J=4.8 Hz and 4.0 Hz, 1H, Het-H), 7.70 (dd, J=3.6 Hz and 1.2 Hz, 1H, Het-H), 8.15 (dd, J=4.8 Hz and 1.2 Hz, 1H, Het-H) and 13.07 (s, 1H, ═NOH).

### Example 12

This example illustrates a method of preparing 2,3-bis(hydroxyimino)-1-(thiophen-2-yl)butan-1-one (14; R¹=2-thiophen-2-yl) in accordance with an embodiment of the invention.

To a solution of 2-(hydroxyimino)-1-phenylbutane-1,3-dione (12) (10 g, 52.3 mmol) and hydroxylamine hydrochloride (4.73 g, 68.0 mmol) in methanol (70 mL) and water (30 mL) was added sodium carbonate (3.66 g, 34.5 mmol) in a minimum amount of water. The reaction mixture was stirred overnight at RT. Excess methanol was removed and the product was extracted with ethyl acetate and solvent was removed under diminished pressure. The crude product was purified on a 340 g Biotage™ silica gel column. Gradient elution with ethyl acetate (10→60%) in hexanes gave the products 14 as white crystals: yield 3.35 g (9.05 mmol, 53%) and 13 (R¹=2-thiophen-2-yl) as a byproduct. ¹H NMR (DMSO-d₆) δ 2.04 (s, 3H, CH₃), 7.23 (dd, J=5.0 Hz and 3.8 Hz, 1H, Het-H), 7.61 (dd, J=3.6 Hz and 1.2 Hz, 1H, Het-H), 8.07 (dd, J=4.8 Hz and 1.2 Hz, 1H, Het-H), 11.85 (s, 1H, ═NOH), 11.92 (s, 1H, ═NOH).

### Example 13

This example illustrates a method of preparing 3-methyl-4-thienoylfuroxan (15; R¹=2-thiophen-2-yl) in accordance with an embodiment of the invention.

To a solution of 2,3-bis(hydroxyimino)-1-(thiophen-2-yl)butan-1-one (14) (1.8 g, 8.48 mmol) in anhydrous diethyl ether (40 mL) and glacial AcOH (1 mL) was added lead (IV) oxide (6.09 g, 25.4 mmol). The resulting mixture was vigorously stirred for 12 h at RT. The reaction mixture was diluted with ether and filtered through celite. The filtrate was evaporated to get crude solid. The crude product was purified on a 100 g Biotage™ silica gel column. Gradient elution with ethyl acetate (5→30%) in hexanes gave the products 15 as white solid: yield 0.606 g (2.88 mmol, 34%). ¹H NMR (CDCl₃) δ 2.46 (s, 3H, CH₃), 7.27 (dd, J=3.8 Hz and 1.0 Hz, 1H, Het-H), 7.89 (dd, J=5 Hz and 1.0 Hz, 1H, Het-H) and 8.39 (dd, J=4.2 Hz and 1.0 Hz, 1H, Het-H); ¹³C NMR (CDCl₃) δ 8.9, 112.2, 129.0, 137.0, 137.3, 140.6, 153.8 and 175.4.

### Example 14

This example illustrates a method of preparing 3-bromomethyl-4-thienoylfuroxan (16; R¹=2-thiophen-2-yl) with an embodiment of the invention.

A mixture of 3-methyl-4-thienoylfuroxan (15) (1.77 g, 8.42 mmol), NBS (5.99 g, 33.7 mmol), and AIBN (0.138 g, 0.842 mmol) in CCl₄ (50 mL) was refluxed for 12 h. Then another portion of NBS (5.99 g, 33.7 mmol) and AIBN (0.138 g, 0.842 mmol) were added and refluxed again for 12 h. The reaction mixture was filtered through Celite™, washed with CCl₄ and evaporated to provide crude solid. The crude product was purified on a 100 g Biotage™ silica gel column. Gradient elution with ethyl acetate (5→30%) in hexanes gave the product 16 as white solid: yield 1.4 g (4.84 mmol, 57.5%). ¹H NMR (CDCl₃) δ 4.62 (s, 2H, CH₂), 7.24 (dd, J=5.4 and 1.0 Hz, 1H, Het-H), 7.88 (dd, J=4.8 and 1.2 Hz, 1H, Het-H) and 8.39 (dd, J=4.0 Hz and 1.2 Hz, 1H, Het-H); ¹³C NMR (CDCl₃) δ 16.4, 111.9, 129.1, 129.8, 137.3, 137.8, 152.3 and 174.6.

### Example 15

This example illustrates a method of preparing 4-thienoylfuroxan-3-methanol (17; R¹=2-thiophen-2-yl) in accordance with an embodiment of the invention.

A mixture of 3-bromomethyl-4-thienoylfuroxan (16) (1.2 g, 4.15 mmol) and sulfuric acid (6 M, 17.3 mL, 104 mmol,) in dioxane (20 mL) was refluxed for 10 h. The reaction mixture was extracted with ethyl acetate; successively washed with water, saturated NaHCO₃ and brine. The crude product was purified on a 100 g Biotage™ silica gel column. Gradient elution with ethyl acetate (12→80%) in hexanes gave the product 17 as white solid: yield 0.62 g (2.74 mmol, 66%). ¹H NMR (DMSO-d₆) δ 4.57 (d, J=6.4 Hz, 2H, CH₂), 5.63 (t, J=6.0 Hz, 1H, OH), 7.39 (dd, J=5.0 Hz and 3.8 Hz, 1H, Het-H), 8.28 (dd, J=3.8 Hz and 1.0 Hz, 1H, Het-H) and 8.32 (dd, J=4.8 Hz and 1.2 Hz, 1H, Het-H).

### Example 16

This example illustrates a method of preparing 3-((N-(t-butyldimethylsilyloxy)-4-methylphenylsulfonamido)methyl)-4-thienoylfuroxan (18; R¹=2-thiophen-2-yl) in accordance with an embodiment of the invention.

To a solution of 4-thienoylfuroxan-3-methanol (17) (0.5 g, 2.21 mmol) N-(t-butyldimethylsilyloxy)-4-methylbenzenesulfonamide (0.61 g, 2.01 mmol), and triphenylphosphine (1.05 g, 4.02 mmol) in toluene (7.5 mL) and THF (2.5 mL) was added dropwise DEAD (0.84 ml, 2.11 mmol) at 0° C. The reaction mixture was allowed to attain room temperature in 2 h. The reaction mixture was diluted with ethyl acetate, successively washed with water, saturated NaHCO₃, brine and dried (MgSO₄). The crude product was purified on a 50 g Biotage™ silica gel column. Gradient elution with ethyl acetate (2→40%) in hexanes gave the product 18 as white solid: yield 0.93 g (1.83 mmol, 91%). ¹H NMR (CDCl₃) δ 0.22 (s, 6H, CH₃), 0.88 (s, 9H, CH₃), 2.49 (s, 3H, CH₃), 4.30 (s, 2H, CH₂) 7.27 (dd, J=5.0 Hz and 3.8 Hz, 1H, Het-H), 7.39 (d, J=8.4 Hz, 2H, Ar—H), 7.76 (d, J=8.4 Hz, 2H, Ar—H), 7.91 (dd, J=5.0 Hz and 1.0 Hz, 1H, Het-H) and 8.31 (dd, J=4.0 Hz and 1.2 Hz, 1H, Het-H); ¹³C NMR (CDCl₃) δ −4.9, 17.9, 21.7, 25.8, 47.8, 110.7, 128.2, 129.1, 129.5, 130.5, 137.5, 137.6, 140.4, 145.6, 153.6 and 175.1.

### Example 17

This example illustrates a method of preparing 4-thienoylfuroxan-3-carbaldehyde oxime (19; R¹=2-thiophen-2-yl) in accordance with an embodiment of the invention.

To a solution of 3-((N-(t-butyldimethylsilyloxy)-4-methylphenylsulfonamido)methyl)-4-thienoylfuroxan (18) (0.1 g, 0.196 mmol) in acetonitrile (2 mL) was added CsF (0.060 g, 0.392 mmol) and stirred at RT for 10 min. The reaction mixture was acidified with 10% HCl and extracted with ethyl acetate. The crude product after evaporation of ethyl acetate was purified on a 10 g Biotage™ silica gel column. Gradient elution with ethyl acetate (6→50%) in hexanes gave the product 19 as white solid: yield 0.022 g (0.092 mmol, 47%). ¹H NMR (DMSO-d₆) δ 7.33-7.36 (m, 1H, Het-H), 7.58 (s, 1H, N═CH), 8.13 (dd, J=4.0 Hz and 1.2 Hz, 1H, Het-H), 8.29 (dd, J=4.8 Hz and 1.2 Hz, 1H, Het-H) and 12.65 (s, 1H, C═NOH).

### Example 18

This example illustrates a method of preparing 4-thienoyl-3-cyanofuroxan (20; R¹=2-thiophen-2-yl) in accordance with an embodiment of the invention.

To a solution of 4-thienoylfuroxan-3-carbaldehyde oxime (19) (0.1 g, 0.42 mmol) in DMF (2 mL) was added dropwise thionyl chloride (0.130 mL, 1.78 mmol) at 0° C. The reaction mixture was allowed to attain RT over 3 h. The reaction mixture was quenched with ice water and extracted with ethyl acetate. The organic layer was successively washed with water, saturated NaHCO₃, brine and dried (Na₂SO₄). Further purification was done using reverse phase (0.05% aq. TFA/MeCN) HPLC to afford the product 20 as white solid: yield 0.073 g (0.33 mmol, 79%). ¹H NMR (CDCl₃) δ 7.30 (dd, J=5.2 and 4.4 Hz, 1H, Het-H), 7.97 (dd, J=4.8 and 1.2 Hz, 1H, Het-H) and 8.41 (dd, J=4.0 Hz and 1.2 Hz, 1H, Het-H); ¹³C-NMR (CDCl₃) δ 95.3, 104.5, 129.3, 137.4, 138.8, 138.9 152.6 and 171.4.

### Example 19

This example illustrates the functional bioactivity of inventive oxadiazole-2-oxide compounds of Formula IV, in accordance with an embodiment, using the TGR enzyme inhibition assay.

Recombinant thioredoxin glutathione reductase (“TGR”) with a fused bacterial-type SECIS element was expressed in Escherichia coli strain BL21(DE3) (Invitrogen, Carlsbad, Calif.) and purified according to the method described in Kuntz et al., PLoS Med., 4, e206 (2007). TGR concentration was determined from the flavin adenine dinucleotide absorption (ε₄₆₃=11.3 mM⁻¹ cm⁻¹).

Test compounds of Formula IV were dissolved in DMSO to produce 10 mM initial stock solutions. The samples were then serially diluted row-wise in 384-well plates in twofold steps for a total of 12 concentrations. Upon completion of the serial dilution protocol, solutions from up to two 384-well plates were transferred to duplicate wells of a 1,536-well compound plate at 7 μL per well. The last two rows of the 1,536-well plate did not contain any test compound and were reserved for placement of positive and negative controls. A Flying Reagent Dispenser (FRD) (formerly Aurora Discovery, presently Beckman-Coulter, San Diego, Calif.) was used to dispense reagents into the assay plates.

Three microliters of reagents (100 μM NADPH in row 32 as no-enzyme control and 100 μM NADPH/15 μM TGR, in rows 1-31) were dispensed into 1,536-well Greiner (Monroe, N.C.) black clear-bottom assay plates. Compounds (23 nL) were transferred via a Kalypsys Pin-Tool equipped with a 1,536-pin array (10 nL slotted pins, V&P Scientific, Palo Alto, Calif.). The plate was incubated for 15 min at room temperature, and then a 1 μL aliquot of 500 μM NADPH was added, immediately followed by a 1 μL aliquot of 15 mM DTNB to start the reaction. The second addition of NADPH is done to minimize the effect of non-inhibitory redox cyclers, that is, compounds that might exhaust NADPH during the 15 min incubation and thus give the erroneous appearance of inhibition. The plate was transferred to a ViewLux™ high-throughput charge-coupled device (CCD) imager (PerkinElmer, Waltham, Mass.) where kinetic measurements (five reads, one read every 2 min) of the 5-thio-2-nitrobenzoic acid (TNB) absorbance were acquired using a 405 nm excitation filter.

For activity calculations, delta values, computed as the difference in absorbance between last and first time points, were used, while the calculated slope, intercept, and the raw time-course data were stored in the database. Screening data were corrected and normalized, and concentration-effect relationships were derived by using in-house software. Percentage activity was computed from the median values of the uninhibited, or neutral, control and the no-enzyme, or 100% inhibited, control, respectively. The results are set forth in Table 1.

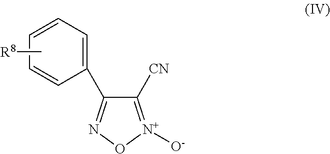

wherein Formula IV is an embodiment of Formula I.

TABLE 1

| Compound | R⁸ | TGR IC₅₀ (μM) | TGR Max. Resp. (μM) |
|---|---|---|---|
| 49* | H | 6.3 | −102 |
| 25 | 4-NO₂ | 2.2 | −107 |
| 32 | 3-CF₃ | 2.5 | −102 |
| 35 | 3-Br, 4-F | 2.8 | −102 |
| 31 | 3-Br | 2.8 | −100 |
| 30 | 3-Cl | 3.5 | −101 |
| 23 | 4-Br | 3.5 | −99 |
| 22 | 4-Cl | 4.0 | −99 |
| 24 | 4-CF₃ | 7.1 | −103 |
| 46 | 3-OH | 7.1 | −96 |
| 21 | 4-F | 7.9 | −103 |
| 34 | 2-OMe | 7.9 | −96 |
| 38 | 3,4,5-(OMe)₃ | 8.9 | −102 |
| 33 | 3-OMe | 8.9 | −102 |
| 26 | 4-OMe | 10.0 | −97 |
| 27 | 4-Me | 11.2 | −98 |
| 28 | 4-Ph | 15.8 | −71 |
| 48 | 4-OH | 17.9 | −64 |
| 55 | 3-NO₂ | 2.2 | −107 |

*Sayed et al., Nature Medicine 14(4), 407-412 (2008)

As is apparent from the results set forth in Table 1, the inventive compounds inhibited thioredoxin glutathione reductase with IC₅₀ values ranging from 2.2 μM to 17.9 μM.

As is further apparent from the results set forth in Table 1, the maximum response of TGR activity at levels denoting full efficacy of the compounds (flat curve asymptotes) was equivalent to levels indicating complete inhibition of TGR activity.

### Example 20

This example illustrates the functional bioactivity of inventive oxadiazole-2-oxide compounds of Formula V, wherein Formula V is an embodiment of Formula I, using the TGR enzyme inhibition assay described in Example 18. The results are set forth in Table 2.

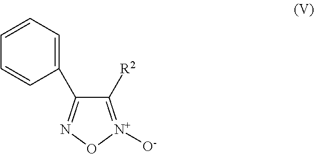

TABLE 2

| Compound | R² | TGR IC₅₀ (μM) | TGR Max. Resp. (μM) |
|---|---|---|---|
| 49* | CN | 6.3 | −102 |
| 50 | CH₃ | >50 | −15 |
| 51 | CH₂OH | 11.2 | −100 |
| 52 | CHO | 0.11 | −105 |

*Sayed et al., Nature Medicine 14(4), 407-412 (2008)

As is apparent from the results set forth in Table 2, inventive compounds 52 and 51 inhibited thioredoxin glutathione reductase with IC₅₀ values of 0.11 μM and 11.2 μM, respectively. Inventive compound 50 inhibited thioredoxin glutathione reductase but was less potent than compounds 52, 51, and 56.

### Example 21

This example illustrates the functional bioactivity of inventive oxadiazole-2-oxide compounds of Formula IV using the ex vivo parasite killing assay.

Test compounds were dissolved in DMSO and added at 10 μM to freshly perfused S. mansoni worms in RPMI 1640 containing 25 mM of HEPES, 150 units/mL of penicillin, 125 μg/mL of streptomycin, and 10% FCS (Cell Grow, Fisher) at pH 7. The time for 100% of the worms to die (“ET₁₀₀”) was determined for each test compound, and the results set forth in Table 3.

TABLE 3

| Compound | R⁸ | Approximate ET₁₀₀ (hours) |
|---|---|---|
| 49* | H | 16-18 |
| 25 | 4-NO₂ | 24 |
| 32 | 3-CF₃ | 24 |
| 35 | 3-Br, 4-F | 18 |
| 30 | 3-Cl | 18 |
| 23 | 4-Br | 16 |
| 24 | 4-CF₃ | 24 |
| 21 | 4-F | 24 |
| 34 | 2-OMe | 48 |
| 38 | 3,4,5-(OMe)₃ | 48 |
| 33 | 3-OMe | >72 |
| 26 | 4-OMe | 48 |
| 27 | 4-Me | 24 |
| 28 | 4-Ph | >72 |
| 48 | 4-OH | 48 |

*Sayed et al., Nature Medicine 14(4), 407-412 (2008)

As is apparent from the results set forth in Table 3, compounds 21, 23, 24, 25, 26, 27, 30, 32, 34, 35, 38, and 48 killed 100% of treated S. mansoni worms within 48 hours or less. Compounds 28 and 33 required over 72 hours to kill 100% of treated S. mansoni worms.

### Example 22

This example illustrates the functional bioactivity of inventive oxadiazole-2-oxide compounds of Formula III, wherein R² is CN, in accordance with an embodiment, using the TGR enzyme inhibition assay described in Example 18. The results are set forth in Table 4.

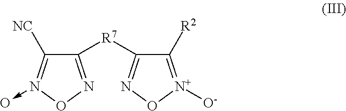

TABLE 4

| | | | TGR Max. Resp. |
|---|---|---|---|
| Compound | R⁷ | TGR IC₅₀ (μM) | (μM) |
| 43 | 1,3-phenylene | 3.5 | −95 |
| 44 | 1,4-phenylene | 1.0 | −98 |
| 45 | 5-F-1,3-phenylene | 0.48 | −95 |
| 53 | thiophen-2,5-diyl | 0.40 | −102 |
| 54 | thiophen-2,4-diyl | 0.35 | −92 |

As is apparent from the results set forth in Table 1, the inventive compounds inhibited thioredoxin glutathione reductase with IC₅₀ values ranging from 2.2 μM to 17.9 μM.

As is further apparent from the results set forth in Table 1, the maximum response of TGR activity at levels denoting full efficacy of the compounds (flat curve asymptotes) was equivalent to levels indicating complete inhibition of TGR activity.

### Example 23

This example illustrates the functional bioactivity of inventive oxadiazole-2-oxide compound of Formula III using the ex vivo parasite killing assay described in Example 20.

Test compounds 43, 44, 45, 53, and 54 were dissolved in DMSO and added at 10 μM to freshly perfused S. mansoni worms in RPMI 1640 containing 25 mM of HEPES, 150 units/mL of penicillin, 125 μg/mL of streptomycin, and 10% FCS (Cell Grow, Fisher) at pH 7. After 48 hours, 100% of the worms were judged to be dead for each of compounds 43, 44, 45, 53, and 54.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of the formula (I): 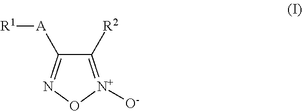
wherein A is selected from the group consisting of a bond, —C(═O)—, —C(═NR³)—, and —C(═NOR³)—,
R¹ is selected from the group consisting of a C₆-C₁₀ aryl group, a heterocycloaryl group, and R⁶, each optionally substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, C₆-C₁₀ heterocycloaryl, 3-cyano-1,2,5-oxadiazol-4-yl-2 oxide, C₁-C₆ haloalkyl, C₁-C₆ dihaloalkyl, C₁-C₆ trihaloalkyl, —NO₂, —OH, —OR⁴, —SH, —SR⁴, —SOR⁴, —SO₂R⁴, —COR⁴, —COOH, —COOR⁴, —CONHR⁴, and —CONHR⁴R⁵,
R² is selected from the group consisting of C₁-C₆ alkyl, C₂-C₆ alkenyl, C₂-C₆ alkynyl, C₃-C₈ cycloalkyl, C₃-C₈ cycloalkenyl, —CH₂OH, —CHO, —COOH, —CONH₂, —C═NR⁵, —C═NOH, —C═NOR⁵, and —CN,
R³, R⁴, and R⁵ are selected from the group consisting of C₁-C₆ alkyl, C₂-C₆ alkenyl, C₂-C₆ alkynyl, C₃-C₈ cycloalkyl, and C₃-C₈ cycloalkenyl, and
R⁶ is methylenedioxyphenyl, 2,3-benzofuranyl, or 2,3-dihydrobenzofuranyl,
with the proviso that when A is a bond and R² is CN or CONH₂, R¹ is not unsubstituted aryl,
or a pharmaceutically acceptable salt thereof.

2. The compound or salt of claim 1, wherein A is a bond or —C(═O)—.

3. The compound or salt of claim 2, wherein R² is selected from the group consisting of —CH₂OH, —CHO, —C═NR⁵, —C═NOH, —C═NOR⁵, and —CN.

4. The compound or salt of claim 3, wherein R² is selected from the group consisting of —CHO, —C═NOH, —C═NOR⁵, and —CN.

5. The compound or salt of claim 4, wherein R² is —CN.

6. The compound or salt of claim 2, wherein R¹ is a C₆-C₁₀ aryl group substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, 3-cyano-1,2,5-oxadiazol-4-yl-2-oxide, C₁-C₆ haloalkyl, C₁-C₆ dihaloalkyl, C₁-C₆ trihaloalkyl, —NO₂, —OH, —OR⁴, —SH, —SR⁴, —COR⁴, —COOR⁴, —CONHR⁴, and —CONHR⁴R⁵.

7. The compound or salt of claim 6, wherein R¹ is a phenyl group substituted by 1, 2, 3, 4, or 5 substituents selected from the group consisting of halo, C₁-C₆ trihaloalkyl, —NO₂, —OH, and —OR⁵.

8. The compound or salt of claim 2, wherein R¹ is a heterocycloaryl group optionally substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, 3-cyano-1,2,5-oxadiazol-4-yl-2 oxide, C₁-C₆ haloalkyl, C₁-C₆ dihaloalkyl, C₁-C₆ trihaloalkyl, —NO₂, —OH, —OR⁴, —SH, —SR⁴, —COR⁴, —COOR⁴, —CONHR⁴, and —CONHR⁴R⁵.

9. The compound or salt of claim 8, wherein R¹ is selected from the group consisting of furan-2-yl, thiophen-2-yl, 2-pyridyl, 3-pyridyl, and 4-pyridyl.

10. The compound or salt of claim 2, wherein A is —C(═O)—.

11-17. (canceled)

18. The compound or salt of claim 1, wherein the compound is selected from the group consisting of 3-cyano-4-(4-fluorophenyl)-1,2,5-oxadiazole-2-oxide, 3-cyano-4-(4-chlorophenyl)-1,2,5-oxadiazole-2-oxide, 3-cyano-4-(4-bromophenyl)-1,2,5-oxadiazole-2-oxide, 3-cyano-4-(4-trifluoromethylphenyl)-1,2,5-oxadiazole-2-oxide, 3-cyano-4-(4-nitrophenyl)-1,2,5-oxadiazole-2-oxide, 3-cyano-4-(4-methoxyphenyl)-1,2,5-oxadiazole-2-oxide, 3-cyano-4-p-tolyl-1,2,5-oxadiazole-2-oxide, 3-cyano-4-(biphenyl-4-yl)-1,2,5-oxadiazole-2-oxide, 3-cyano-4-(4-(prop-2-ynyloxy)phenyl)-1,2,5-oxadiazole-2-oxide, 3-cyano-4-(3-nitrophenyl)-1,2,5-oxadiazole-2-oxide, 3-cyano-4-(3-chlorophenyl)-1,2,5-oxadiazole-2-oxide, 3-cyano-4-(3-bromophenyl)-1,2,5-oxadiazole-2-oxide, 3-cyano-4-(3-trifluoromethylphenyl)-1,2,5-oxadiazole-2-oxide, 3-cyano-4-(3-methoxyphenyl)-1,2,5-oxadiazole-2-oxide, 3-cyano-4-(3-hydroxyphenyl)-1,2,5-oxadiazole-2-oxide, 3-cyano-4-(2-methoxyphenyl)-1,2,5-oxadiazole-2-oxide, 3-cyano-4-(3-bromo-4-fluorophenyl)-1,2,5-oxadiazole-2-oxide, 3-cyano-4-(4-chloro-3-nitrophenyl)-1,2,5-oxadiazole-2-oxide, 3-cyano-4-(3,5-bis(trifluoromethylphenyl))-1,2,5-oxadiazole-2-oxide, 3-cyano-4-(3,4,5-trimethoxyphenyl)-1,2,5-oxadiazole-2-oxide, 3-cyano-4-(benzo[d][1,3]dioxol-5-yl)-1,2,5-oxadiazole-2-oxide, and 3-cyano-4-(naphthalene-2-yl)-1,2,5-oxadiazole-2-oxide.

19. The compound or salt of claim 1, wherein the compound is selected from the group consisting of 3-cyano-4-(furan-2-yl)-1,2,5-oxadiazole-2-oxide, 3-cyano-4-(5-nitrofuran-2-yl)-1,2,5-oxadiazole-2-oxide, and 3-cyano-4-(thiophen-2-yl)-1,2,5-oxadiazole-2-oxide.

20. The compound or salt of claim 1, wherein the compound is 3-cyano-4-thienoyl-furoxan.

21. The compound or salt of claim 1 having formula (III): 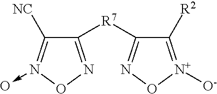
wherein R⁷ is selected from the group consisting of a C₆-C₁₀ aryl group and a heterocycloaryl group, and wherein each is optionally further substituted by 1, 2, 3, 4, or 5 substituents selected from the group consisting of halo, C₁-C₆ alkyl, C₂-C₆ alkenyl, C₂-C₆ alkynyl, C₃-C₈ cycloalkyl, C₃-C₈ cycloalkenyl, C₆-C₁₀ aryl, C₆-C₁₀ heterocycloaryl, C₁-C₆ haloalkyl, C₁-C₆ dihaloalkyl, C₁-C₆ trihaloalkyl, —NO₂, —OH, —OR⁴, —SH, —SR⁴, —SOR⁴, —SO₂R⁴, —COR⁴, —COOH, —COOR⁴, —CONHR⁴, and —CONHR⁴R⁵, and
R² is selected from the group consisting of C₁-C₆ alkyl, C₂-C₆ alkenyl, C₂-C₆ alkynyl, C₃-C₈ cycloalkyl, C₃-C₈ cycloalkenyl, —CH₂OH, —CHO, —COOH, —CONH₂, —C═NR⁵, —C═NOH, —C═NOR⁵, and —CN.

22. The compound or salt of claim 21, wherein R⁷ is optionally further substituted by 1, 2, 3, 4, or 5 substituents selected from the group consisting of halo, C₁-C₆ trihaloalkyl, nitro, hydroxy, and —OR⁴, and
R² is —CN.

23. The compound or salt of claim 22, wherein the compound is selected from the group consisting of 4,4′-(1,3-phenylene)bis(3-cyano-1,2,5-oxadiazole) 2-oxide, 4,4′-(1,4-phenylene)bis(3-cyano-1,2,5-oxadiazole) 2-oxide, and 4,4′-(5-fluoro-1,3-phenylene)bis(3-cyano-1,2,5-oxadiazole) 2-oxide.

24. The compound or salt of claim 22, wherein the compound is selected from the group consisting of 4,4′-(thiophen-2,4-diyl)bis(3-cyano-1,2,5-oxadiazole 2-oxide) and 4,4′-(thiophen-2,5-diyl)bis(3-cyano-1,2,5-oxadiazole 2-oxide).

25-29. (canceled)

30. The compound or salt of claim 2, wherein A is a bond.

31. The compound or salt of claim 3, wherein A is a bond.

32. The compound or salt of claim 6, wherein A is a bond.

33. The compound or salt of claim 8, wherein A is a bond.

34. The compound or salt of claim 9, wherein A is a bond.

35. The compound or salt of claim 3, wherein A is —C(═O)—.

36. The compound or salt of claim 6, wherein A is —C(═O)—.

37. The compound or salt of claim 8, wherein A is —C(═O)—.

38. The compound or salt of claim 9, wherein A is −C(═O)—.

39. A pharmaceutical composition comprising the compound or salt of claim 1 and a pharmaceutically acceptable carrier.

40. A method for treating schistomasiasis in a mammal comprising administering an effective amount of the compound or salt of claim 1 to a mammal afflicted therewith.

41. The method of claim 40, wherein the mammal is a human.

42. A method of inhibiting thioredoxin glutathione reductase (TGR) of S. mansoni in a mammal invaded by said S. mansoni comprising administering an effective amount of a compound or salt of claim 1.
